(19) United States
(12) Patent Application Publication
(10) Pub. No.: US 2025/0160939 A1
(43) Pub. Date: May 22, 2025

(54) A SYSTEM FOR FORMING A FISTULA BETWEEN TWO ADJACENT VESSELS

(71) Applicant: CLEARSTREAM TECHNOLOGIES LIMITED, Enniscorthy, County Wexford (IE)

(72) Inventors: Michael Whelan, Enniscorthy, County Wexford (IE); Pat Byrne, Enniscorthy, County Wexford (IE); John O'Shea, Enniscorthy, County Wexford (IE)

(21) Appl. No.: 18/873,425

(22) PCT Filed: Jun. 10, 2022
(86) PCT No.: PCT/EP2022/065822

§ 371 Date: Dec. 10, 2024

(51) Int. Cl. A61B 18/14; A61B 17/11; A61B 18/00

(52) U.S. Cl. CPC A61B 18/1492; A61B 2017/1107; A61B 2018/00702; A61B 2018/00827

(57) ABSTRACT

A system for forming a fistula between two adjacent vessels. The system comprises a first catheter comprising a housing with an electrode; a second catheter comprising a backstop; an energy source for supplying radiofrequency energy to the electrode; a current sensor for measuring the current flowing to the electrode; and a waveform detector coupled to the current sensor for determining a phase state of the electrode based on a waveform of the measured current.

### TECHNICAL FIELD

The present disclosure relates to a system for forming a fistula between two adjacent vessels and a method of forming a fistula between two adjacent vessels.

### BACKGROUND

A fistula denotes a passageway formed between two internal organs. Forming a fistula between two blood vessels can have one or more beneficial functions. For example, the formation of a fistula between an artery and a vein may provide access to the vasculature for haemodialysis patients. Specifically, forming a fistula between an artery and a vein allows blood to flow quickly between the vessels while bypassing the capillaries. Needles, catheters, or other cannulas may then be inserted into the blood vessels near the fistula to draw blood from the circulatory system, pass it through a dialysis machine, and return it to the body. The quickened flow provided by the fistula may provide for effective haemodialysis.

In other instances, a fistula may be formed between two veins to form a veno-venous fistula. Such a veno-venous fistula may be used to treat portal venous hypertension. Specifically, cirrhosis or other liver diseases may cause increased resistance to flow from the portal veins draining from the intestine to the liver. This increased resistance may cause massive dilation of blood vessels, which may rupture spontaneously. To help prevent this undesirable outcome, a fistula may be formed between a portal vein and one of the major branches, thereby lowering venous pressure in the portal vein.

The creation of fistulas also plays an important role in the treatment of peripheral arterial disease (PAD). Peripheral arterial disease can result from the occlusion of arteries in the legs and lower extremities such as the feet. Typically, such occlusion is brought about by atherosclerosis, whereby calcified plaque deposited on the walls of an arterial vessel causes narrowing and blockage of the vessel lumen. Treatment options for PAD may include deep vein arterialisation (DVA), whereby blood flow is routed from the diseased artery to a nearby deep vein in order to supply the extremity with blood. Another possible treatment is an endovascular bypass procedure, whereby blood flow is routed out of the artery and back into the artery by a conduit that circumvents the blockage. Both of these procedures require the formation of one or more fistulas between an artery and a vein in order to redirect the blood flow past the occlusion in the artery.

In the above scenarios, the fistulas often must be formed in vessels walls which are highly calcified. The calcification increases the thickness of the vessel walls and makes it more difficult to cut through the wall and form the fistula.

US 2012/0302935 A1 and US 2017/0202616 A1 disclose systems and methods for forming a fistula using an endovascular approach. These systems comprise a first catheter having a housing with an electrode and a second catheter having a housing with a backstop. However, these systems do not provide the user with information about whether the fistula formation process was successful or not. Furthermore, these systems are also limited in calcified vessels by the distance the electrode can fire through the venous/arterial walls.

There is hence a need in the art for a new system for forming a fistula, which can provide a user with more information about the state of the fistula formation process.

Furthermore, there is a need in the art for a system which can detect if a vessel wall is calcified, to allow the fistula forming process to be adjusted and achieve a more effective fistula formation.

### SUMMARY

In a first aspect of the present disclosure, there is provided a system for forming a fistula between two adjacent vessels. The system comprises a first catheter comprising a housing with an electrode; a second catheter comprising a backstop; an energy source for supplying radiofrequency energy to the electrode; a current sensor for measuring the current flowing to the electrode; and a waveform detector coupled to the current sensor for determining a phase state of the electrode based on a waveform of the measured current.

In some embodiments, this system may be able to provide a user with more information about the state of the fistula formation process, which may allow the fistula formation process to be adjusted to result in more effective fistula formation.

The waveform detector may comprise an oscilloscope.

In some embodiments, this may allow for effective waveform detection.

The oscilloscope may be configured to display the waveform of the measured current.

In some embodiments, this may allow a visualisation of the waveform for the benefit of a user.

The waveform detector may be configured to measure the average peak amplitude of the waveform of the measured current over a number of cycles and determine a dry-out phase state if the average peak amplitude rises above a first threshold.

In some embodiments, this may provide an effective means for detecting the dry-out phase state.

Throughout this disclosure, the term “average peak amplitude” is used to refer to the peak amplitude of a wave measured and averaged over a number of cycles, for example, in the range of 5 to 100 cycles.

The waveform detector may be further configured to determine a plasma phase state if the average peak amplitude is below a second threshold.

In some embodiments, this may provide an effective means for detecting the plasma phase state.

The second threshold may be lower than the first threshold.

The waveform detector may be further configured to determine a pre-plasma phase state when the average peak amplitude is between the first and second threshold.

In some embodiments, this may provide an effective means for detecting the pre-plasma phase state.

The waveform detector may be configured to determine a time taken to reach the dry-out phase state.

The waveform detector may be configured to detect whether calcium is present in a vessel wall based on the time taken to reach the dry-out phase state.

In some embodiments, this may allow the system to detect the presence of calcium in the vessel wall to allow the fistula forming process to be adjusted and achieve a more effective fistula formation.

The waveform detector may be configured to determine that too much calcium is present in the vessel wall to form a fistula, if the time taken to reach the dry-out phase state is below a first threshold time.

In some embodiments, this may allow the system to detect a position in the vessel where it is not feasible to form a fistula due to excess calcium in the vessel wall.

The waveform detector may be configured to determine that a fistula was successfully formed, if the time taken to reach the dry-out phase state is above a second threshold time.

In some embodiments, this may allow for the determination of a successful fistula formation.

The second threshold time may be longer than the first threshold time.

The waveform detector may be configured to activate an indicator or alarm when the electrode reaches the dry-out phase state.

In some embodiments, this may alert a user who can adjust the fistula formation process to reduce or prevent damage to the electrode and vessel wall.

The system may further comprise a processor coupled to the energy source and the waveform detector.

The processor may be configured to turn off the energy source when it receives a signal from the waveform detector that the electrode has reached the dry-out phase state.

In some embodiments, this may prevent damage to the electrode to allow multiple fistulas to be formed with the same electrode in the same procedure.

The processor may be configured to reduce the power of the energy source when it receives a signal from the waveform detector that the electrode has reached the dry-out phase state.

In some embodiments, this may prevent damage to the electrode to allow multiple fistulas to be formed with the same electrode in the same procedure.

The system may further comprise a voltage sensor for measuring the voltage of the electrode, wherein the waveform detector is coupled to the voltage sensor.

In some embodiments, this may provide a user with more information about the state of the fistula formation process, which may allow the fistula formation process to be adjusted to result in more effective fistula formation.

The backstop may be non-conductive.

In some embodiments, this may prevent arcing between the electrode and the backstop.

The backstop may be at least partly made from a ceramic material.

In some embodiments, this may help the backstop to better withstand the heat and plasma generated by the electrode.

The energy source may be configured to supply a constant power signal to the electrode.

The electrode may be disposed at least partially within the housing.

The electrode may comprise a distal end, a proximal end and an intermediate portion therebetween for contacting a vessel wall and forming the fistula.

The electrode may have a radially expanded configuration, and a radially contracted configuration.

In some embodiments, this may allow the electrode to more effectively form a fistula in the radially expanded configuration and to more effectively be introduced into a patient's body in the radially contracted configuration.

Throughout this disclosure, the term “radially expanded configuration” is used to refer to a configuration where the electrode extends further f from the housing than in a “radially contracted configuration”.

The proximal end of the electrode may be fixed and the distal end of the electrode may be moveable with respect to the housing.

In some embodiments, this may allow the electrode to more easily move between the radially expanded configuration and the radially contracted configuration.

The electrode may comprise a leaf spring.

In some embodiments, this may allow the electrode to more easily move between the radially expanded configuration and the radially contracted configuration without breaking.

Throughout this disclosure, the term “leaf spring” is used to refer to a flexible curved strip of material which can be bent but will regain its original shape when released.

The electrode may have a convex shape.

In some embodiments, this may result in more effective fistula formation.

The backstop may be a recessed backstop which may have a complementary shape to the electrode.

In some embodiments, this may result in better compression of the vessel walls between the electrode and the backstop and therefore more effective fistula formation.

The first catheter and the second catheter may each comprise one or more magnets positioned to align the electrode with the backstop.

In some embodiments, this may result in accurate and precise alignment of the electrode with the backstop.

The system may further comprise a grounding pad for contacting the body of a patient.

In a second aspect of the present disclosure, there is provided a method of forming a fistula between two adjacent vessels. The method comprises introducing a first catheter having a housing into an electrode a first vessel; introducing a second catheter having a backstop into a second adjacent vessel; supplying a radiofrequency energy signal to the electrode of the first catheter; measuring the current through the electrode with a current sensor and sending the measurements to a waveform detector; and determining a phase state of the electrode based on a waveform of the measured current.

Determining a phase state of the electrode may comprise measuring the average amplitude of the waveform of the measured current over a number of cycles and determining: a dry-out phase state if the average peak amplitude rises above a first threshold; a plasma phase state if the average peak amplitude is below a second threshold, wherein the second threshold is lower than the first threshold; and a pre-plasma phase state when the average peak amplitude is between the first and second threshold.

The method may further comprise determining a time taken to reach the dry-out phase state.

The waveform detector may be configured to determine that too much calcium is present in the vessel wall to form a fistula, if the time taken to reach the dry-out phase state is below a first threshold time, or determine that a fistula was successfully formed, if the time taken to reach the dry-out phase state is above a second threshold time.

### BRIEF DESCRIPTION OF THE DRAWINGS

To enable better understanding of the present disclosure, and to show how the same may be carried into effect, reference will now be made, by way of example only, to the accompanying drawings, in which:

FIG. 1 shows a first catheter and a second catheter for forming a fistula.

FIG. 2 illustrates a schematic diagram of a system for forming a fistula according to the present disclosure.

FIG. 3A shows an idealised waveform of the current during a fistula forming process.

FIG. 3B shows a graph of the average amplitude of the current during a fistula formation process.

### DETAILED DESCRIPTION

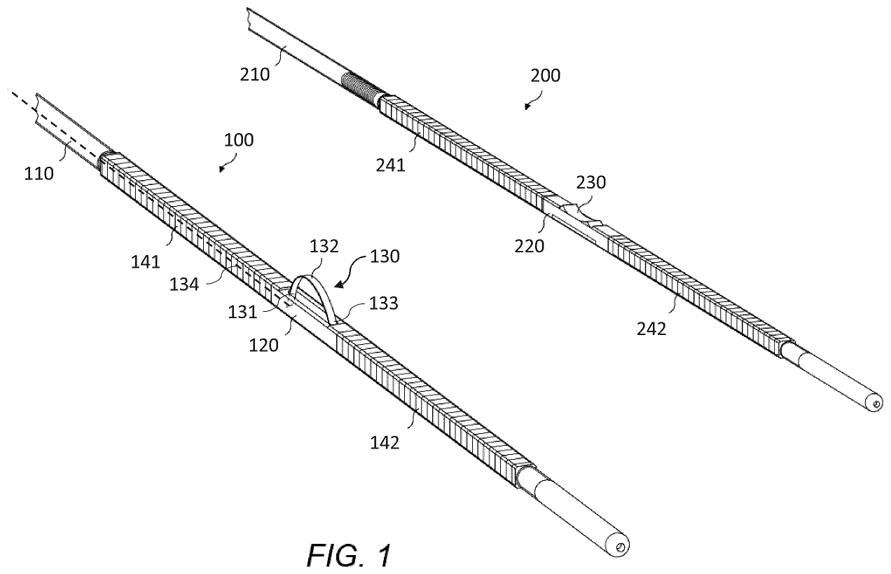

FIG. 1 shows an example of a catheter system for forming a fistula. The system comprises a first catheter 100 and a second catheter 200 which can be used together to form a fistula between two vessels.

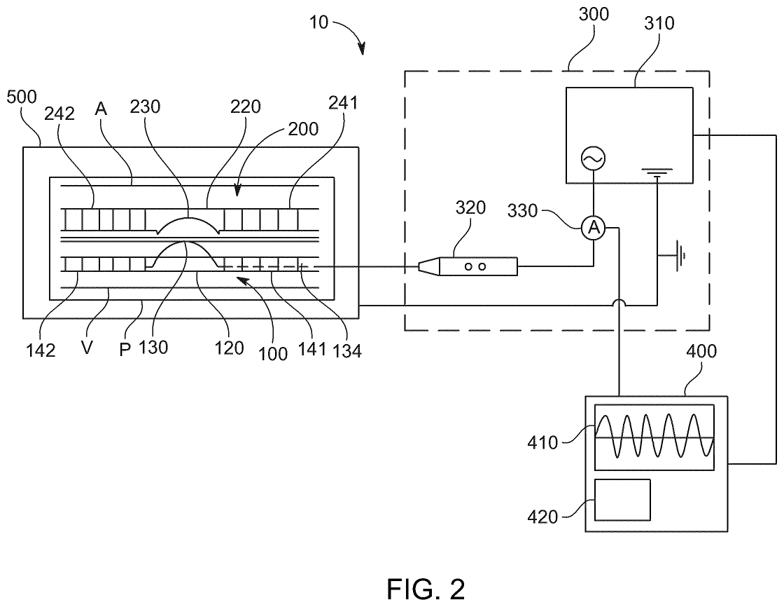

The first catheter 100 comprises a catheter shaft 110 and a housing 120 disposed at the distal end of the shaft 110. An electrode 130 is partially disposed in the housing 120 and may extend out of an opening of the housing 120. The electrode 130 may have a proximal portion 131, an intermediate portion 132 and a distal portion 133. A connecting element 134 may be connected to the proximal portion 131 and may extend along the shaft 110 of the first catheter 100. The proximal end of the connecting element 134 may be connected to an RF energy source, for example an ESU pencil 420 (see FIG. 2), to allow RF energy to be supplied to the electrode 130. The proximal portion 131 may be fixed to the housing 120, for example, with a clamping mechanism or an adhesive. The intermediate portion 132 may extend out of the opening of the housing 120 and come into contact with the vessel wall for forming the fistula. The intermediate portion 132 may have a convex shape which extends away from the housing 120. The distance between the top of the intermediate portion 132 and the housing is the height of the electrode 130. The distal portion 133 may not be fixed such that it can move longitudinally relative to the housing 120. This may allow the electrode 130 to move between a radially contracted configuration and a radially expanded configuration. In the radially expanded configuration, the electrode 130 extends radially further from the housing 120 than in the radially contracted configuration.

The electrode 130 may be in the form of a ribbon wire and may be made from a number of suitable materials, such as one or more refractory metals. For example, the electrode 130 may comprise tungsten, molybdenum, niobium, tantalum, rhenium, or combinations and alloys thereof. The housing 120 may be made from a non-conductive ceramic material which can withstand the heat and plasma generated by the electrode 130.

The first catheter 100 may also comprise a proximal set of magnets 141 and a distal set of magnets 142 which are disposed proximally and distally of the housing 120, respectively.

The second catheter 200 also comprises a catheter shaft 210 and a second housing 220 having a backstop 230 disposed at the distal end of the shaft 210. The backstop 230 may have a concave portion which is shaped complimentary to the convex portion of the electrode 130. The second housing 220 and the backstop 230 may also be made from a non-conductive ceramic material to withstand the heat and plasma generated by the electrode 130. The second catheter 200 also comprises a proximal set of magnets 241, disposed proximally of the housing 220, and a distal set of magnets 242, disposed distally of the housing 220.

In order to form a fistula between two vessels, such as artery and vein, the first catheter 100 may be introduced into the venous system through an access site and advanced to the treatment site where a fistula is to be formed. The second catheter 200 may be introduced into the arterial system through a second access site and also advanced to the treatment site where the fistula is to be formed. The first catheter 100 may be advanced to the treatment site inside a sheath. The sheath may compress the electrode 130 such that it is in the radially contracted configuration. This allows the first catheter 100 to more easily advance through the vessel due to the lower profile. The sheath may then be removed once the first catheter is in position at the treatment site which causes the electrode to move to the expanded position.

The first catheter 100 and second catheter 200 may be advanced to the treatment site from the same direction or opposite directions.

Once the first catheter 100 and the second catheter 200 are positioned at the treatment site from the same direction (as shown in FIG. 2, for example), the proximal set of magnets 141 of the first catheter 100 will be attracted to the proximal set of magnets 241 of the second catheter 200 and align themselves with each other. Similarly, the distal set of magnets 142 of the first catheter 100 will be attracted to the distal set of magnets 242 of the second catheter 200 and these sets of magnets will align with each other. This will result in the electrode 130 becoming aligned with the concave portion of the backstop 230. The backstop 230 is configured to compress the vessel walls in a localised region for ablation by the electrode 130 of the first catheter 100. The sets of magnets may also have the effect of pulling the artery A and vein V closer together.

Radiofrequency (RF) energy may then be supplied to the electrode 130 which causes the electrode 130 to heat up and generate a plasma. The plasma causes rapid dissociation of molecular bonds in organic compounds and allows the electrode 130 to cut through the venous and arterial vessel walls until it hits the backstop 230 to form the fistula.

However, the first catheter 100 is limited in the distance it can cut through the vessel walls. If the vessel walls are calcified, this can substantially increase the thickness of the vessel walls and means that it may not be possible to cut through the vessel walls without deforming and damaging the electrode.

FIG. 2 shows a schematic diagram of a system 10 for forming a fistula. The system 10 comprises the first catheter 100, the second catheter 200, an electrosurgical unit (ESU) 300, a current sensor 330 and a waveform detector 400.

The first catheter 100 is shown disposed in a vein V and the second catheter 200 disposed in an adjacent artery A of a patient P, prior to the process of forming an arteriovenous fistula. The electrode 130 is in a radially expanded configuration and aligned with the backstop 230 through the attraction and alignment of the proximal set of magnets 141, 241 and the distal set of magnets 142, 242, respectively.

The electrosurgical unit 300 comprises an energy source for supplying radiofrequency energy to the electrode 130, in the form of a generator 310. An output of the generator 310 may be connected to an ESU pencil 320, which is in turn connected to the connecting and element 134 electrode 130. Radiofrequency current can flow from the generator 310 output via the ESU pencil 320 and the connecting element 134 to the electrode 130. The ESU pencil 320 may have a button or switch for activating the RF electrode 130.

The current sensor 330 may be positioned at any point between the generator 310 and the electrode 130, for example at the output of the generator as shown in FIG. 2, to measure the current flowing through the electrode 130. The current sensor 330 may be a current probe, for example, and is connected to the waveform detector 400.

The waveform detector 400 may be an oscilloscope which can detect, interpret and visualize the signals from the current sensor 330. The waveform detector 400 may comprise a processor 420 which can determine a waveform of the measured current and measure a number of characteristics of the waveform, for example, the peak amplitude of the wave and the average peak amplitude of the wave over a number of cycles. The processor 420 may use logic saved in memory to use the measured characteristics of the waveform to control operation of the system 10. The waveform detector 400 may comprise a display 410 for visualizing the waveform of the measured current and communicating that information to a user. The waveform detector 400 may also be connected to the generator 310 such that the processor 420 can send commands to the generator 310. While the processor 420 and display 410 are shown as part of the waveform detector 400, they may be separate from the waveform detector 400.

The system 10 may further comprise a grounding pad 500 which may be in contact with the body of the patient P and connected to a ground terminal of the generator 310. The grounding pad 500 safely returns current from the patient P back to the ground terminal of the generator 310.

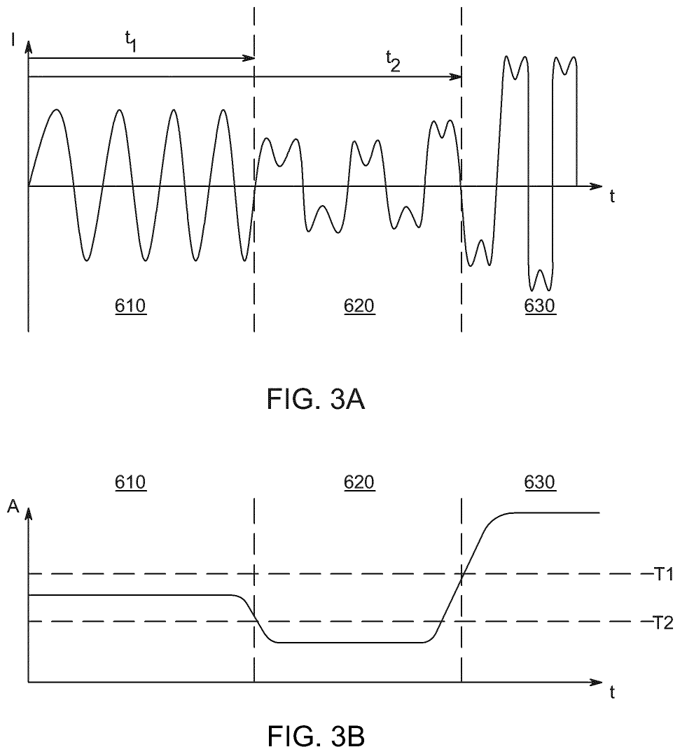

FIG. 3A shows an idealised graph of a waveform of the current of the electrode 130 during a fistula formation process. The y-axis represents the current I flowing through the electrode 130. The x-axis represents the time t elapsed during the fistula formation process. During a fistula formation process, the electrode 130 will go through three distinct phases, a pre-plasma phase 610, a plasma phase 620 and a dry-out phase 630.

When the first catheter 100 and the second catheter 200 are positioned at the treatment site and aligned with each other, as shown in FIG. 2, the fistula formation process commences. A constant power sinusoidal RF signal is supplied from the electrosurgical unit 400 to the electrode 130.

As shown in FIG. 3A, the electrode 130 initially enters a pre-plasma phase 610, where the electrode 130 heats up and the current flowing through the electrode 130 is in the form of a sine wave with a single peak and a constant peak amplitude.

Once the electrode 130 is heated up sufficiently, it will heat up the surrounding tissue and start to generate a plasma. The electrode 130 will enter the plasma phase 620. During the plasma phase 620, the plasma generated by the electrode 130 causes rapid dissociation of molecular bonds in organic compounds and allows the electrode 130 to cut through the venous and arterial vessel walls. The peak amplitude of the current decreases, and the waveform of the current starts to develop dual or twin peaks.

Once the fistula has been formed and the electrode 130 is hitting the backstop 230, or if the vessel wall is excessively calcified such that no plasma can be effectively generated, the electrode 130 enters the dry-out phase 630. During the dry-out phase 630, the peak amplitude of the current waveform increases beyond the peak amplitude of the pre-plasma phase 610 and the twin peaks remain. If the electrode 130 is continued to be operated in the dry-out phase, it can cause the electrode 130 to overheat and deform, causing damage to the electrode 130 and possible damage to the vessel walls of the patient P.

The system 10 of FIG. 2 is therefore configured to detect the pre-plasma phase 610, plasma phase 620 and dry-out phase 630 of the electrode 130 and communicate this information to the user during the fistula formation process, for example via the display 420 of the waveform detector 400 to allow the user to take action and adjust the fistula formation process to allow for more effective fistula formation.

In order to determine the phase state of the electrode 130, the processor 420 of the waveform detector 400 measures the average peak amplitude of the current waveform over a number of cycles. For example, the peak amplitude may be measured and averaged over 5 to 100 cycles, to obtain a value for the average peak amplitude.

FIG. 3B shows a graph of the average peak amplitude A during the fistula formation process. The y-axis represents the average peak amplitude A, while the x-axis represents the time t elapsed during the fistula formation process.

In the pre-plasma phase 610, the average peak amplitude is at a constant level between a first threshold value T1 and a second threshold value T2, where the second threshold value T2 is lower than the first threshold value T1. Once the plasma phase 620 is reached, the average peak amplitude decreases below the second threshold value T2. When the dry-out phase 630 is reached, the average peak amplitude increases above the first threshold T1.

The processor 420 of the waveform detector 400 continually measures the average peak amplitude of the current during the fistula formation process. If the average peak amplitude decreases below the second threshold T2, the waveform detector detects that the electrode 130 is in the plasma phase 620. Once the average peak amplitude rises above the first threshold T1, the waveform detector 400 detects that the electrode 130 is in the dry-out phase 630. As can be seen, during the pre-plasma phase 610, the average peak amplitude of the current is between the first and second thresholds T1 and T2. To this end, the first and second thresholds T1 and T2 can be selected based on a predetermined average peak amplitude value during the pre-plasma phase 610 and also the predetermined peak amplitude values during the plasma and dry-out phases 620 and 630.

The processor 420 of the waveform detector 400 can also measure the time taken t1 to reach the plasma phase 620 and the time taken t2 to reach the dry-out phase 630. The waveform detector 400 can then communicate this information to the user via the display 410, or via an alarm or indicator. This may allow a user, for example, to stop the fistula formation process when the dry-out phase 630 is reached to prevent damage to the electrode 130.

The waveform detector 400 can also make a determination of whether a fistula was successfully formed or whether there is excess calcium in the vessel wall which does not allow a fistula to be successfully formed, based on the time taken t2 to reach the dry-out phase 630. The time taken t1 to reach the plasma-phase 620 is generally the same for the same electrode 130 and RF power settings. Small variations may be seen depending on the surface contact of the electrode 130 with the tissue. Therefore, if the time t2 is above a first threshold time, this means that the electrode 130 has been able to form an effective plasma and has had time to cut through the vessel walls to reach the backstop before entering the dry-out phase 630. In that case, the waveform detector 400 may determine that a fistula was successfully formed.

If the time t2 is below a second threshold time, which may be the same or smaller than the first threshold time, the electrode 130 was only able to form a plasma for a short time due to the heavy calcification in the vessel walls which prevents effective plasma formation, before reaching the dry-out phase 630. In that case, the electrode 130 was not able to cut through the heavy calcified vessel walls and the waveform detector 400 may determine that no successful fistula was formed.

Alternatively, the processor 420 of the waveform detector 40 can calculate the length of the plasma phase 620 by subtracting the time taken t2 to reach the dry-out phase 630 from the time taken t1 to reach the plasma phase 620. The same determination of whether a successful fistula has been formed or whether the length of the plasma phase is above or below a certain threshold time.

The waveform detector 400 may communicate the information about whether a fistula was successfully formed or whether there is excess calcium in the vessel wall, which did not allow a fistula to be successfully formed, to the user via the display 410, for example.

If a determination is made that excess calcium is present in the vessel wall, the user may then move the first electrode 100 and the second electrode 200 to a different position where less calcium is present in the vessel wall and repeat the fistula formation process.

As shown in FIG. 2, the waveform detector 400 may also be connected to the generator 310. The processor 420 can send commands to the generator 310. For example, the processor 420 can be configured to send a signal to turn off the power of the generator 310 when the waveform detector has made a determination that the dry-out phase 630 has been reached. Alternatively, the processor 420 can be configured to send a signal to reduce the power of the generator 310 when the waveform detector has made a determination that the dry-out phase 630 has been reached. Turning off or reducing the power from the generator 310 when the dry-out phase is reached will reduce or prevent damage to the electrode 130. This allows multiple fistulas to be formed with the same electrode 130 during a procedure.

Various modifications will be apparent to those skilled in the art.

The first catheter 100 may not comprise any proximal magnets 141 and/or distal magnets 142.

The second catheter 200 may not comprise any proximal magnets and/or distal magnets.

The shape of the electrode 130 is not limited to a convex shape, but could be any other suitable shape, such as for example V-shaped or rectangular-shaped.

The electrode 130 is not limited to a ribbon wire, but may be any other type of suitable wire, for example, a cylindrical wire or oval wire.

The electrode may not have a radially expanded configuration and a radially contracted configuration. The electrode may only have one constant configuration.

The backstop 230 of the second catheter 220 is not limited to a concave shape but may be any suitable shape. For example, the backstop 230 may be recessed or protruding and could have a concave, convex or rectangular shape.

The housing 120 of the first catheter 100 and the housing 220 of the second catheter 200 is not limited to a ceramic material and may be made from any suitable material which can withstand the heat and plasma generated by the electrode 130.

The system 10 may not comprise an ESU pencil 320. The generator 310 may be connected directly to the first catheter 100. In that case, the first catheter 100 may have a catheter handle with an RF activation button.

The waveform detector 400 may not be an oscilloscope but may be any other suitable type of waveform detector.

The waveform detector 400 may not be connected to the generator 310.

The waveform detector 400 may not comprise a display 410.

The system 10 may not comprise a grounding pad 500.

The energy source for supplying radiofrequency energy is not limited to a generator 310 but may be any suitable type of radiofrequency energy source.

The system 10 may not comprise a second catheter 200.

The waveform detector 400 may not measure the average peak amplitude of the waveform of the measured current but may determine the phase state of the electrode differently, for example, by detecting the twin peaks in the waveform.

All of the above are fully within the scope of the present disclosure and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A system for forming a fistula between two adjacent vessels, the system comprising:
a first catheter comprising a housing with an electrode;
a second catheter comprising a backstop;
an energy source for supplying radiofrequency energy to the electrode;
a current sensor for measuring the current flowing to the electrode; and
a waveform detector coupled to the current sensor for determining a phase state of the electrode based on a waveform of the measured current.

2. The system of claim 1, wherein the waveform detector comprises an oscilloscope.

3. The system of claim 2, wherein the oscilloscope is configured to display the waveform of the measured current.

4. The system of claim 1, wherein the waveform detector is configured to measure the average peak amplitude of the waveform of the measured current over a number of cycles and determine a dry-out phase state if the average peak amplitude rises above a first threshold.

5. The system of claim 4, wherein the waveform detector is further configured to determine a plasma phase state if the average peak amplitude is below a second threshold.

6. The system of claim 5, wherein the second threshold is lower than the first threshold.

7. The system of claim 5, wherein the waveform detector is further configured to determine a pre-plasma phase state when the average peak amplitude is between the first and second threshold.

8. The system of claim 4, wherein the waveform detector is configured to determine a time taken to reach the dry-out phase state.

9. The system of claim 8, wherein the waveform detector is configured to detect whether calcium is present in a vessel wall based on the time taken to reach the dry-out phase state.

10. The system of claim 8, wherein the waveform detector is configured to determine that too much calcium is present in the vessel wall to form a fistula, if the time taken to reach the dry-out phase state is below a first threshold time.

11. The system of claim 8, wherein the waveform detector is configured to determine that a fistula was successfully formed, if the time taken to reach the dry-out phase state is above a second threshold time.

12. The system of claim 11, wherein the second threshold time is longer than the first threshold time.

13. The system of claim 4, wherein the waveform detector is configured to activate an indicator or alarm when the electrode reaches the dry-out phase state.

14. The system of claim 4, further comprising a processor coupled to the energy source and the waveform detector.

15. The system of claim 14, wherein the processor is configured to turn off the energy source when it receives a signal from the waveform detector that the electrode has reached the dry-out phase state.

16. The system of claim 14, wherein the processor is configured to reduce the power of the energy source when it receives a signal from the waveform detector that the electrode has reached the dry-out phase state.

17-28. (canceled)

29. A method of forming a fistula between two adjacent vessels, the method comprising:
introducing a first catheter having a housing an electrode into a first vessel;
introducing a second catheter having a backstop into a second adjacent vessel;
supplying a radiofrequency energy signal to the electrode of the first catheter;
measuring the current through the electrode with a current sensor and sending the measurements to a waveform detector; and
determining a phase state of the electrode based on a waveform of the measured current.

30. The method of claim 29, wherein determining a phase state of the electrode comprises measuring the average amplitude of the waveform of the measured current over a number of cycles and determining:
a dry-out phase state if the average peak amplitude rises above a first threshold;
a plasma phase state if the average peak amplitude is below a second threshold, wherein the second threshold is lower than the first threshold; and
a pre-plasma phase state when the average peak amplitude is between the first and second threshold.

31. The method of claim 30, wherein further comprising determining a time taken to reach the dry-out phase state.

32. The method of claim 31, wherein the waveform detector is configured to determine that too much calcium is present in the vessel wall to form a fistula, if the time taken to reach the dry-out phase state is below a first threshold time, or determine that a fistula was successfully formed, if the time taken to reach the dry-out phase state is above a second threshold time.
